(19) United States
(12) Patent Application Publication
(10) Pub. No.: US 2021/0100440 A1
(43) Pub. Date: Apr. 8, 2021

(54) ENDOSCOPE APPARATUS, OPERATION METHOD OF ENDOSCOPE APPARATUS, AND INFORMATION STORAGE MEDIUM

(71) Applicant: OLYMPUS CORPORATION, Tokyo (JP)

(72) Inventors: Hiroki TANIGUCHI, Tokyo (JP); Jumpei TAKAHASHI, Tokyo (JP)

(73) Assignee: OLYMPUS CORPORATION, Tokyo (JP)

(21) Appl. No.: 17/126,522

(22) Filed: Dec. 18, 2020

Related U.S. Application Data
(63) Continuation of application No. PCT/JP2018/025210, filed on Jul. 3, 2018.

(51) Int. Cl. A61B 1/06; A61B 1/05; A61B 1/00; A61B 1/307; H04N 5/225; H04N 9/04

(52) U.S. Cl. CPC A61B 1/0638; A61B 1/051; A61B 1/00009; H04N 2005/2255; A61B 1/00045; H04N 5/2256; H04N 9/0451; A61B 1/307

(57) ABSTRACT

An endoscope apparatus includes: an illumination device emitting first to third illumination light; an imaging device capturing an image using return light from a subject; and a processor including hardware. The processor generates a display image based on first to third images captured with the first to third light emitted. A first absorbance difference (difference in absorbance of β-carotene between the first and second illumination light) is smaller than a second absorbance difference (difference in absorbance of metmyoglobin between the first and second illumination light). A peak wavelength of the third illumination light differs from peak wavelengths of the first and second illumination light. Based on the first to third images, the processor generates the display image that displays a thermally denatured muscle layer, a fat layer, and a muscle layer that is not thermally denatured of the subject, in an identifiable manner from each other.

### CROSS REFERENCE TO RELATED APPLICATION

This application is a continuation of International Patent Application No. PCT/JP2018/025210, having an international filing date of Jul. 3, 2018, which designated the United States, the entirety of which is incorporated herein by reference.

### BACKGROUND

A procedure of transurethrally resecting a bladder tumor using an endoscope apparatus (transurethral resection of the bladder tumor; TUR-Bt) is widely known. In TUR-Bt, a tumor is resected in the state where the bladder is filled with a perfusion solution. The bladder wall is thinly stretched due to the perfusion solution. As the procedure is done in this state, TUR-Bt involves a risk of perforation.

The bladder wall consists of three layers of a mucosa layer, a muscle layer, and a fat layer in this order from inside to outside. Hence, displaying the layers in such a manner as to allow for easy identification of each layer would help avoid perforation.

For in-vivo observation and treatment using an endoscope apparatus, methods for highlighting a specific object through image processing are widely known. For example, Japanese Unexamined Patent Application Publication No. 2016-067775 discloses a method for highlighting information on blood vessels located at a specific depth based on image signals taken by emission of light within a specific wavelength band. International Publication No. WO2013/115323 discloses a method for highlighting a fat layer by emission of illumination light within a plurality of wavelength bands taking into account an absorption characteristic of β-carotene.

### SUMMARY

In accordance with one of some aspect, there is provided an endoscope apparatus comprising:

an illumination device emitting first illumination light, second illumination light, and third illumination light;

an imaging device capturing an image using return light, from a subject, based on light emitted from the illumination device; and

a processor including hardware,

the processor being configured to generate a display image on the basis of a first image captured with the first illumination light emitted, a second image captured with the second illumination light emitted, and a third image captured with the third illumination light emitted,

a first absorbance difference being smaller than a second absorbance difference, the first absorbance difference being a difference between an absorbance of β-carotene at a peak wavelength of the first illumination light and an absorbance of β-carotene at a peak wavelength of the second illumination light, the second absorbance difference being a difference between an absorbance of metmyoglobin at the peak wavelength of the first illumination light and an absorbance of metmyoglobin at the peak wavelength of the second illumination light,

a peak wavelength of the third illumination light differing from the peak wavelength of the first illumination light and the peak wavelength of the second illumination light,

the processor generating, based on the first image, the second image, and the third image, the display image that displays a thermally denatured muscle layer of the subject, a fat layer of the subject, and a muscle layer of the subject that is not thermally denatured, in a manner allowing for identification of the layers from each other.

In accordance with one of some aspect, there is provided an operation method of an endoscope apparatus, the method comprising:

emitting first illumination light, second illumination light, and third illumination light;

capturing an image using return light, from a subject, based on emission of the first illumination light, the second illumination light, and the third illumination light; and

generating a display image on the basis of a first image captured with the first illumination light emitted, a second image captured with the second illumination light emitted, and a third image captured with the third illumination light emitted,

a first absorbance difference being smaller than a second absorbance difference, the first absorbance difference being a difference between an absorbance of β-carotene at a peak wavelength of the first illumination light and an absorbance of β-carotene at a peak wavelength of the second illumination light, the second absorbance difference being a difference between an absorbance of metmyoglobin at the peak wavelength of the first illumination light and an absorbance of metmyoglobin at the peak wavelength of the second illumination light,

a peak wavelength of the third illumination light differing from the peak wavelength of the first illumination light and the peak wavelength of the second illumination light,

the generating the display image comprising generating, based on the first image, the second image, and the third image, the display image that displays a thermally denatured muscle layer of the subject, a fat layer of the subject, and a muscle layer of the subject that is not thermally denatured, in a manner allowing for identification of the layers from each other.

In accordance with one of some aspect, there is provided a non-transitory information storage medium storing a program, the program causing a computer to execute steps comprising:

causing an illumination device to emit first illumination light, second illumination light, and third illumination light;

capturing an image using return light, from a subject, based on light emitted from the illumination device; and

generating a display image on the basis of a first image captured with the first illumination light emitted, a second image captured with the second illumination light emitted, and a third image captured with the third illumination light emitted,

a first absorbance difference being smaller than a second absorbance difference, the first absorbance difference being a difference between an absorbance of β-carotene at a peak wavelength of the first illumination light and an absorbance of β-carotene at a peak wavelength of the second illumination light, the second absorbance difference being a difference between an absorbance of metmyoglobin at the peak wavelength of the first illumination light and an absorbance of metmyoglobin at the peak wavelength of the second illumination light,

a peak wavelength of the third illumination light differing from the peak wavelength of the first illumination light and the peak wavelength of the second illumination light,

the step of generating the display image comprising generating, based on the first image, the second image, and the third image, the display image that displays a thermally denatured muscle layer of the subject, a fat layer of the subject, and a muscle layer of the subject that is not thermally denatured, in a manner allowing for identification of the layers from each other.

### BRIEF DESCRIPTION OF THE DRAWINGS

FIGS. 1A and 1B explain TUR-Bt.

FIG. 2 illustrates a configuration example of an endoscope apparatus.

FIGS. 3A and 3B illustrate an example of spectral characteristics of illumination light in accordance with a first embodiment, and FIG. 3C explains absorbance of each pigment.

FIG. 4 is a flowchart explaining an operation of the endoscope apparatus.

FIG. 5 is a flowchart explaining processing in a white light observation mode.

FIG. 6 is a flowchart explaining processing in a special light observation mode in accordance with the first embodiment.

FIG. 7 illustrates an example of spectral characteristics of a color filter of an image sensor.

FIG. 8 illustrates another configuration example of the endoscope apparatus.

FIGS. 9A and 9B illustrate an example of spectral characteristics of illumination light in accordance with a second embodiment, and FIG. 9C explains absorbance of each pigment.

FIG. 10 is a flowchart explaining processing in a special light observation mode in accordance with the second embodiment.

FIGS. 11A and 11B illustrate an example of spectral characteristics of illumination light in accordance with a third embodiment, and FIG. 11C explains absorbance of each pigment.

FIG. 12 is a flowchart explaining processing in a special light observation mode in accordance with the third embodiment.

### DESCRIPTION OF EXEMPLARY EMBODIMENTS

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

Exemplary embodiments are described below. Note that the following exemplary embodiments do not in any way limit the scope of the content defined by the claims laid out herein. Note also that all of the elements described in the present embodiment should not necessarily be taken as essential elements.

### 1. Method of Exemplary Embodiments

First, a description will be given of a method in accordance with exemplary embodiments. While the below description takes an example of TUR-Bt, the method of the embodiments may be applied to other situations that require identification of a fat layer and a thermally denatured muscle layer. In other words, the method of the embodiments may be applied to other procedures on the bladder, such as transurethral resection of bladder tumor in one-piece (TUR-BO), and may also be applied to observations and procedures on portions other than the bladder.

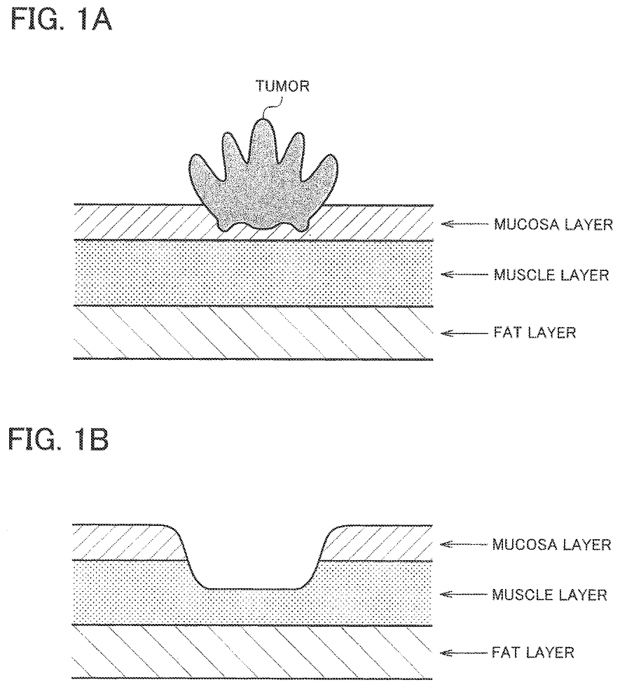

FIGS. 1A and 1B explain TUR-Bt. FIG. 1A schematically illustrates an example of a portion of the bladder wall having a tumor thereon. The bladder wall consists of three layers of a mucosa layer, a muscle layer, and a fat layer, from inside to outside in this order. The tumor stays in the mucosa layer at its relatively early stage, but gradually invades deeper layers including the muscle layer and the fat layer as it develops. By way of example, FIG. 1A illustrates the tumor that has not invaded the muscle layer.

FIG. 1B schematically illustrates an example of a portion of the bladder wall with the tumor resected therefrom by TUR-Bt. In TUR-Bt, at least a portion of the mucosa layer around the tumor is resected. For example, the mucosa layer and a portion of the muscle layer near the mucosa layer are resected. The resected tissue is subject to pathological diagnosis, by which the nature of the tumor and how deep the tumor has grown into the bladder wall are examined. When the tumor is a non-muscle invasive cancer as illustrated in FIG. 1A, the tumor is completely resectable by TUR-Bt, depending on its pathological condition. In other words, TUR-Bt is a procedure that combines diagnosis and treatment.

In view of completely resecting a relatively early stage tumor that has not invaded the muscle layer, resecting the bladder wall up to its relatively deep layer is of importance in the case of TUR-Bt. For example, it is desirable to resect the bladder wall up to an intermediate portion of the muscle layer so that the mucosa layer around the tumor does not remain unremoved. Meanwhile, during TUR-Bt, the bladder wall is being thinly stretched due to a perfusion solution. Hence, resecting the bladder wall excessively up to its deep layer increases a risk of perforation. For example, it is desirable not to resect the fat layer.

To enable an appropriate resection by TUR-Bt, identification of the muscle layer and the fat layer is important. In a typical observation using white light, the muscle layer assumes a whitish or reddish color while the fat layer assumes a yellowish color, and thus these two layers would seem to be identifiable based on their colors. However, TUR-Bt uses an electrosurgical knife to resect a tumor, and this may cause thermal denaturation of the muscle layer. An absorption characteristic of the muscle layer is changed by conversion of myoglobin contained in the muscle layer into metmyoglobin. As a result, the thermally denatured muscle layer assumes a yellowish color, making identification between the fat layer and the thermally denatured muscle layer difficult.

International Publication No. WO2013/115323 discloses a method for displaying the fat layer in a highlighting manner, but the method does not take into consideration color similarity between the fat layer and the thermally denatured muscle layer. As such, conventional methods have difficulty in identifying the fat layer and the thermally denatured muscle layer, and thus may fail to ensure appropriate procedures.

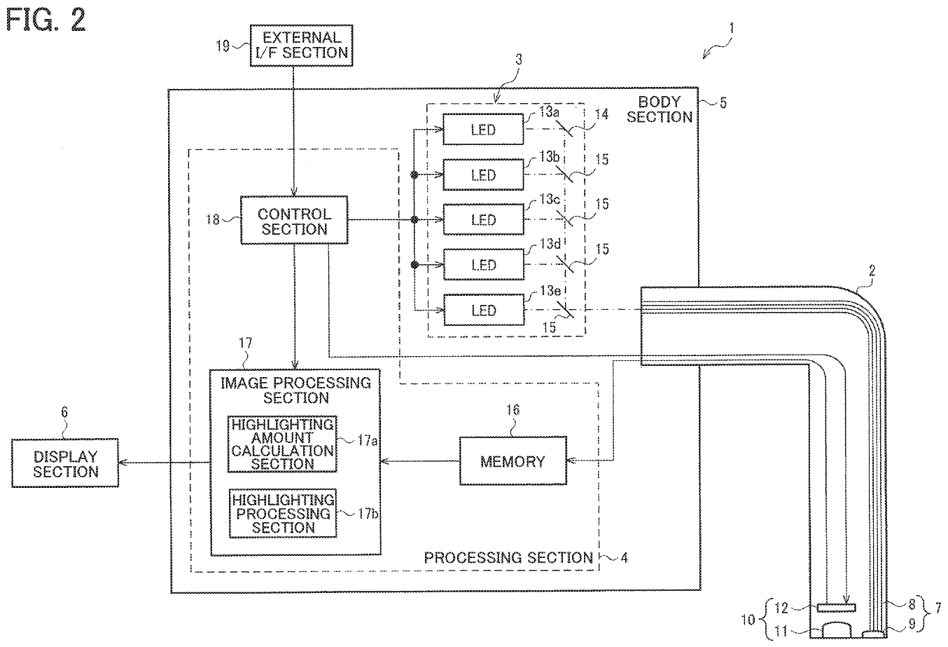

As shown in FIG. 2, an endoscope apparatus 1 in accordance with the embodiments may include an illumination section 3, an imaging section 10, and an image processing section 17. The illumination section 3 emits a plurality of kinds of illumination light including first illumination light, second illumination light, and third illumination light (which may be hereinafter called first light, second light, and third light, respectively). The imaging section 10 captures images using return light, from a subject, based on light emitted from the illumination section 3. The image processing section 17 generates a display image based on a first image captured with the first light emitted, a second image captured with the second light emitted, and a third image captured with the third light emitted.

The first light, the second light, and the third light satisfy the following characteristics. A first absorbance difference is smaller than a second absorbance difference, where the first absorbance difference is a difference between an absorbance of β-carotene at a peak wavelength of the first light and an absorbance of β-carotene at a peak wavelength of the second light, and the second absorbance difference is a difference between an absorbance of metmyoglobin at the peak wavelength of the first light and an absorbance of metmyoglobin at the peak wavelength of the second light. A peak wavelength of the third light differs from the peak wavelength of the first light and the peak wavelength of the second light. The peak wavelength refers to a wavelength at which intensity of the respective light becomes the largest. The absorbance difference as referred to herein is assumed to have a positive value, which is for example a differential absolute value between two absorbances.

β-carotene is a pigment abundant in the fat layer, and metmyoglobin is a pigment abundant in the thermally denatured muscle layer. Since β-carotene has a relatively small absorbance difference between the first light and the second light, correlation between signal values of the first image and the second image is relatively high in a region capturing the fat layer. Since, on the other hand, metmyoglobin has a relatively large absorbance difference between the first light and the second light, correlation between signal values of the first image and the second image is relatively low in a region capturing the thermally denatured muscle layer. Thus, the use of the two kinds of light, chosen in consideration of the absorption characteristics of the pigments contained in the fat layer and the thermally denatured muscle layer, allows for display of the fat layer and the thermally denatured muscle layer in an easily identifiable manner from each other. Preferably, the first absorbance difference has a value that is small enough to be distinctively different from the second absorbance difference, and for example, the difference between the first absorbance difference and the second absorbance difference is equal to or larger than a predetermined threshold. For example, the first absorbance difference is smaller than a first threshold Th1, and the second absorbance difference is larger than a second threshold Th2. For example, the first threshold Th1 is a positive value close to zero, and the second threshold Th2 is larger than the first threshold Th1. More preferably, the absorbance of β-carotene at the peak wavelength of the first light is substantially the same as the absorbance of β-carotene at the peak wavelength of the second light. However, values of the first absorbance difference and the second absorbance difference are only required to be different to the extent that the first absorbance difference and the second absorbance difference are clearly distinguishable from each other, and specific values may be modified in various ways.

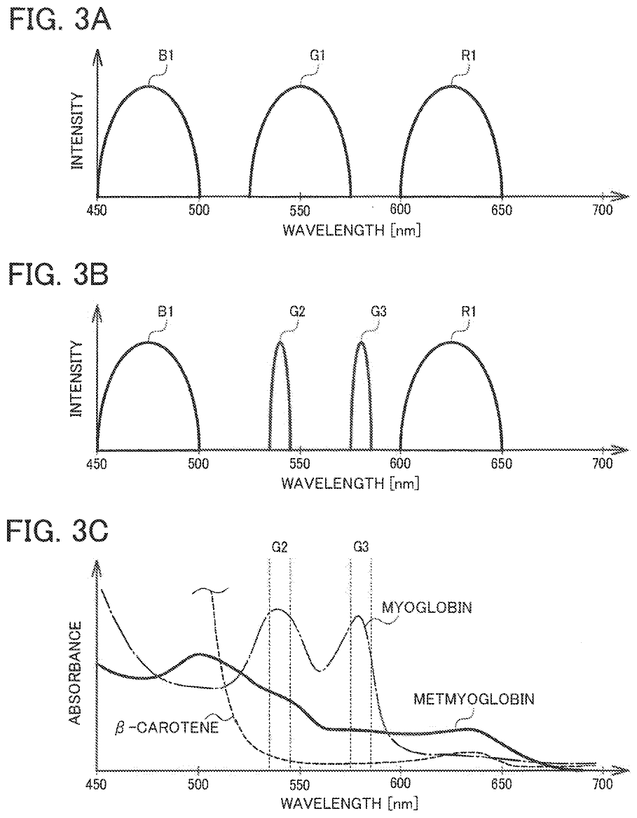

As will be described later with reference to FIG. 3C, absorbance characteristics of β-carotene and metmyoglobin have been known. Thus, it might seem possible to determine which of β-carotene or metmyoglobin is dominant, by referring to signal values of a single image captured by use of single light without making a comparison between two images captured by use of two kinds of light. For example, at a peak wavelength of light G2 (described later), absorbance of metmyoglobin is relatively high while absorbance of β-carotene is relatively low. Thus, in a G2 image obtained by emission of the light G2, it might seem that a region with relatively small signal values (pixel values) could be determined as the thermally denatured muscle layer and a region with relatively large signal values could be determined as the fat layer. However, concentration of pigments contained in an object can vary among objects. Thus it is not easy to set a predetermined threshold that enables determination such that a region with signals smaller than the threshold in an image is the thermally denatured muscle layer and a region with signals larger than the threshold in the image is the fat layer. In other words, accuracy in identification between the fat layer and the thermally denatured muscle layer may be low if the determination relies only on signal values of an image obtained by emission of single light.

In contrast, the method of the embodiments emits two kinds of light and makes identification using the first image and the second image. Comparison of results of emitting the two kinds of light on the same object eliminates the influence of variation in pigment concentration among objects. As a result, this method enables more accurate identification processing as compared to the determination using signal values of a single image.

A captured image may contain an image of object(s) that is/are neither the fat layer nor the thermally denatured muscle layer. In the case of TUR-Bt, a captured image also contains an image of the mucosa layer and the muscle layer that is not thermally denatured. In the following description, the thermally denatured muscle layer is explicitly stated as such, and the simple term “muscle layer” refers to the muscle layer that is not thermally denatured. Both of the mucosa layer and the muscle layer are rich in myoglobin as a pigment. In an observation using white light, the mucosa layer, which has a relatively high myoglobin concentration, is displayed in a more reddish color while the muscle layer, which has a relatively low myoglobin concentration, is displayed in a more whitish color.

Although the first light and the second light have characteristics suitable for identification between the fat layer and the thermally denatured muscle layer, they are not intended to identify other objects different from both of these layers. In this regard, the illumination section 3 of the embodiments emits the third light whose peak wavelength is different from the peak wavelengths of the first light and the second light. This allows for identification of any object that is rich in a pigment different from both of β-carotene and metmyoglobin. Specifically, emitting the third light makes it possible to avoid erroneous highlighting of the mucosa layer or the muscle layer in a highlighting process to increase visibility of the thermally denatured muscle layer.

Preferably, a third absorbance difference is smaller than the second absorbance difference, where the third absorbance difference is a difference between an absorbance of myoglobin at the peak wavelength of the first light and an absorbance of myoglobin at the peak wavelength of the second light. Specifically, the absorbance of myoglobin at the peak wavelength of the first light is substantially the same as the absorbance of myoglobin at the peak wavelength of the second light.

If the first light and the second light have the above characteristics, a region with relatively low correlation between signal values of the first image and the second image can be determined as corresponding to the thermally denatured muscle layer. In other words, a region with relatively high correlation between signal values of the first image and the second image can be determined as corresponding to the fat layer, the muscle layer, or the mucosa layer. As only the region corresponding to the thermally denatured muscle layer can be extracted from a captured image based on the first image and the second image, the method of the embodiments can appropriately highlight the thermally denatured muscle layer while leaving the other regions unhighlighted. For example, when a highlighting process is performed on an entire image as in an example using expressions (1) and (2) given later, the method can greatly change pixel values of a region corresponding to the thermally denatured muscle layer while making relatively small changes to pixel values of a region corresponding to the fat layer, the muscle layer, or the mucosa layer. A specific example of the case where the third absorbance difference is smaller than the second absorbance difference will be described later in first and second embodiments.

However, the third absorbance difference need not necessarily be smaller than the second absorbance difference. In other words, the absorbance of myoglobin at the peak wavelength of the first light need not necessarily be substantially the same as the absorbance of myoglobin at the peak wavelength of the second light, and any absorption characteristics of myoglobin may be used.

As described above, while the fat layer and the thermally denatured muscle layer have similar yellowish colors, the mucosa layer and the muscle layer have colors different from a yellowish color. Hence, through a color determination process, the image processing section 17 can distinguish a region that is determined as either the fat layer or the thermally denatured muscle layer from a region that is determined as an object other than these layers. The image processing section 17 extracts a region that is either the fat layer or the thermally denatured muscle layer from a captured image as preprocessing, and performs a highlighting process based on the first image and the second image only on the detected region. This allows the mucosa layer and the muscle layer to be excluded from highlighting targets at the preprocessing phase. As the first light and the second light are only required to enable identification between the fat layer and the thermally denatured muscle layer, there is no need to consider the absorption characteristic of myoglobin, which allows for a flexible choice of peak wavelengths and wavelength bands of the first light and the second light. This will be detailed later in a third embodiment.

### 2. First Embodiment

Now a description will be given of a first embodiment. A description will be first given of a configuration of the endoscope apparatus 1 with reference to FIG. 2, and a description of processing details will follow. Some modifications will also be described.

2.1 System Configuration Example

FIG. 2 illustrates a system configuration example of the endoscope apparatus 1. The endoscope apparatus 1 includes an insertion section 2, a body section 5, and a display section 6. The body section 5 includes the illumination section 3 connected to the insertion section 2, and a processing section 4.

The insertion section 2 is a portion inserted into a living body. The insertion section 2 includes an illumination optical system 7 that emits light input from the illumination section 3 toward an object, and an imaging section 10 that captures an image of reflected light from the object. Specifically, the imaging section 10 is an imaging optical system.

The illumination optical system 7 includes a light guide cable 8 that guides the light incident from the illumination section 3 to a distal end of the insertion section 2, and an illumination lens 9 that diffuses the light to illuminate the object. The imaging section 10 includes an objective lens 11 that focuses the light emitted by the illumination optical system 7 and reflected by the object, and an image sensor 12 that captures an image of the light focused by the objective lens 11. The image sensor 12 may be implemented by any of various sensors including charge coupled device (CCD) sensors and complementary MOS (CMOS) sensors. Analog signals sequentially output from the image sensor 12 are converted into digital images by an A/D conversion section (not shown). The A/D conversion section may be included either in the image sensor 12 or in the processing section 4.

The illumination section 3 includes a plurality of light emitting diodes (LEDs) 13a-13e each emitting light in a different wavelength band, a mirror 14, and dichroic mirrors 15. Light emitted from each of the plurality of LEDs 13a-13e is made incident into the same light guide cable 8 by the mirror 14 and the dichroic mirrors 15. FIG. 2 illustrates five LEDs, but this is merely exemplary and the number of LEDs is not limited to five. For example, the illumination section 3 may have three or four LEDs as described later. As another alternative, the illumination section 3 may have six or more LEDs.

FIGS. 3A and 3B illustrate spectral characteristics of the plurality of LEDs 13a-13e. In FIGS. 3A and 3B, the horizontal axis represents wavelength, and the vertical axis represents intensity of the emitted light. The illumination section 3 of the present embodiment includes three LEDs respectively emitting light B1 in a blue wavelength band, light G1 in a green wavelength band, and light R1 in a red wavelength band. For example, the wavelength band of B1 is 450-500 nm, the wavelength band of G1 is 525-575 nm, and the wavelength band of R1 is 600-650 nm. The wavelength band of the respective light refers to a wavelength range in which the respective illumination light has intensity at or above a predetermined threshold. However, the wavelength bands of B1, G1, and R1 are not limited to the above and may be modified in various ways such that e.g., the blue wavelength band is 400-500 nm, the green wavelength band is 500-600 nm, and the red wavelength band is 600-700 nm.

The illumination section 3 of the present embodiment further includes two LEDs respectively emitting narrowband light G2 and G3 in a green wavelength band. In the present embodiment, the first light corresponds to G2, and the second light corresponds to G3. That is, the first light is narrowband light with a peak wavelength within a range of 540 nm±10 nm, and the second light is narrowband light with a peak wavelength within a range of 580 nm±10 nm. The narrowband light as referred to herein is light having a narrower wavelength band than the RGB light (B1, G1, and R1 in FIG. 3A), which is used for capturing a white light image. For example, each of G2 and G3 has a half-value width of several nanometers to several tens of nanometers.

FIG. 3C illustrates absorption characteristics of β-carotene, metmyoglobin, and myoglobin. In FIG. 3C, the horizontal axis represents wavelength, and the vertical axis represents absorbance.

β-carotene contained in the fat layer has a flat absorption characteristic within a wavelength band above 530 nm. Myoglobin contained in the muscle layer has similar absorbance peaks at 540 nm and 580 nm. Metmyoglobin contained in the thermally denatured muscle layer has different absorbances at 540 nm and 580 nm.

If G2 and G3 are set to the wavelengths shown in FIG. 3B, the absorbance of β-carotene within the wavelength band of G2 is substantially the same as the absorbance of β-carotene within the wavelength band of G3, and the absorbance of myoglobin within the wavelength band of G2 is substantially the same as the absorbance of myoglobin within the wavelength band of G3. Note that the absorbance of β-carotene within the wavelength band of G2 refers to an absorbance of β-carotene at a peak wavelength of G2 for example, and the absorbance of β-carotene within the wavelength band of G3 refers to an absorbance of β-carotene at a peak wavelength of G3 for example. This holds for myoglobin. Hence, in a region containing a large amount of β-carotene or myoglobin, there is a small difference between signal values (pixel values or luminance values) of a G2 image obtained by emission of G2 and a G3 image obtained by emission of G3.

On the other hand, metmyoglobin has a higher absorbance within the wavelength band of G2 than within the wavelength band of G3. Hence, in a region containing metmyoglobin, signal values of the G2 image obtained by emission of G2 are smaller than those of the G3 image obtained by emission of G3, so that the G2 image is darker than the G3 image in that region.

The processing section 4 includes a memory 16, an image processing section 17, and a control section 18. The memory 16 stores image signals acquired by the image sensor 12 for each wavelength of the illumination light. The memory 16 is, for example, a semiconductor memory such as a static random-access memory (SRAM) and a dynamic random-access memory (DRAM), but may also be a magnetic storage device or an optical storage device.

The image processing section 17 performs image processing on the image signals stored in the memory 16. This image processing includes a highlighting process based on the plurality of image signals stored in the memory 16 and a process of generating a combined display image by allocating the image signals to each of a plurality of output channels. The plurality of output channels in this embodiment is comprised of three channels of an R channel, a G channel, and a B channel, but may be alternatively comprised of three channels of a Y channel, a Cr channel, and a Cb channel, or of any other channel configuration.

The image processing section 17 includes a highlighting amount calculation section 17a and a highlighting processing section 17b. The highlighting amount calculation section 17a is a highlighting amount calculation circuit, for example. The highlighting processing section 17b is a highlighting processing circuit, for example. The highlighting amount as referred to herein is a parameter to determine a degree of highlighting in a highlighting process. In the example using the expressions (1) and (2) given below, the highlighting amount is a parameter not less than 0 and not more than 1, and a smaller value of the parameter causes a larger change in signal values. In other words, in the example given below, the highlighting amount calculated by the highlighting amount calculation section 17a is a parameter whose decrease in value increases the degree of highlighting. However, various modifications to the highlighting amount are possible, such as using a parameter whose increase in value increases the degree of highlighting.

The highlighting amount calculation section 17a calculates the highlighting amount based on correlation between the first image and the second image. More specifically, the highlighting amount calculation section 17a calculates the highlighting amount used for a highlighting process, based on correlation between the G2 image captured by emission of G2 and the G3 image captured by emission of G3. The highlighting processing section 17b performs a highlighting process on a display image based on the highlighting amount. The highlighting process as referred to herein is a process that enables easier identification of the fat layer and the thermally denatured muscle layer than before the highlighting process is done. The display image in the present embodiment refers to an image output from the processing section 4 and displayed by the display section 6. The image processing section 17 may perform other processing on the images acquired from the image sensor 12. For example, the image processing section 17 may execute known processing, such as a white balance process and a noise reduction process, as preprocessing or postprocessing for the highlighting process.

The control section 18 synchronizes the imaging timing of the image sensor 12, the lighting timing of the LEDs 13a-13e, and the image processing timing of the image processing section 17. The control section 18 is a control circuit or a controller, for example.

The display section 6 sequentially displays the display images output from the image processing section 17. In other words, the display section 6 displays a video that consists of the display images as frame images. The display section 6 is a liquid crystal display or an electro-luminescence (EL) display, for example.

An external I/F section 19 is an interface that allows a user to perform an input operation or the like on the endoscope apparatus 1. In other words, the external I/F section 19 may be an interface for operating the endoscope apparatus 1 or an interface for making operational setting for the endoscope apparatus 1. For example, the external I/F section 19 may include a mode switching button for switching observation modes and an adjustment button for adjusting parameters for image processing.

The endoscope apparatus 1 of the present embodiment may be configured as follows. The endoscope apparatus 1 (the processing section 4 in a narrow sense) may include a memory storing information and a processor configured to operate based on the information stored in the memory. The information may include programs and various data, for example. The processor may perform image processing including the highlighting process and controls emission by the illumination section 3. The highlighting process is a process of determining the highlighting amount based on the first image (G2 image) and the second image (G3 image) and highlighting a given image based on the highlighting amount. For example, the image to be highlighted is an R1 image that is allocated to the R output channel, though various modifications are possible.

For example, the processor may implement functions of the respective sections either by individual hardware or integrated hardware. For example, the processor may include hardware, and the hardware may include at least one of a digital signal processing circuit and an analog signal processing circuit. For example, the processor may be composed of one or more circuit devices mounted on a circuit board or may be composed of one or more circuit elements. The circuit device is an integrated circuit (IC), for example. The circuit element is a resistor or a capacitor, for example. The processor may also be a central processing unit (CPU), for example. The processor is, however, not limited to the CPU and may be any of various processors including a graphics processing unit (GPU) and a digital signal processor (DSP). The processor may also be a hardware circuit including an application specific integrated circuit (ASIC). The processor may include an amplifier circuit or a filter circuit that processes analog signals. The memory may be a semiconductor memory such as an SRAM and a DRAM or may be a register. The memory may also be a magnetic storage device such as a hard disk device or an optical storage device such as an optical disc device. For example, the memory stores computer-readable instructions, and functions of the respective sections in the processing section 4 are implemented as the processes by the processor executing the instructions. These instructions may be an instruction set included in a program or may be instructions that cause operations of the hardware circuit included in the processor.

The sections in the processing section 4 of the present embodiment may be implemented as modules of a program running on the processor. For example, the image processing section 17 is implemented as an image processing module. The control section 18 is implemented as a control module configured to perform various controls including synchronization of the emission timing of the illumination light and the imaging timing of the image sensor 12.

The program for implementing the processes performed by the respective sections in the processing section 4 of the present embodiment may be, for example, stored in an information storage device that is a computer-readable medium. For example, the information storage device may be implemented as an optical disk, a memory card, a hard disk drive (HDD), or a semiconductor memory. The semiconductor memory is a read-only memory (ROM), for example. This information storage device may be the memory 16 shown in FIG. 2 or may be one different from the memory 16. The processing section 4 performs various processes in the present embodiment based on the program stored in the information storage device. In other words, the information storage device stores the program for causing a computer to function as each section of the processing section 4. The computer is a device including an input device, a processing section, a storage section, and an output section. The program causes the computer to execute the processing in each section of the processing section 4.

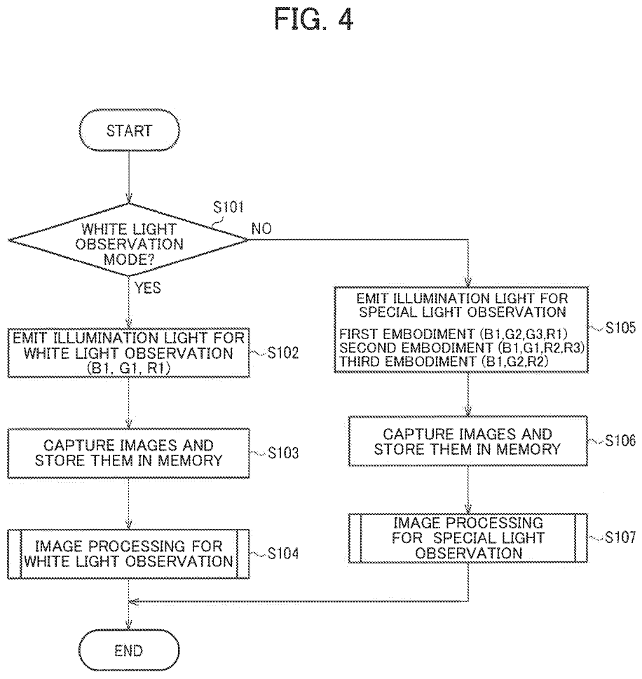

In other words, the method of the present embodiment may be applied to a program that causes a computer to execute steps of causing the illumination section 3 to emit the first light, the second light, and the third light; capturing an image using return light, from a subject, based on light emitted from the illumination section 3; and generating a display image on the basis of the first image captured with the first light emitted, the second image captured with the second light emitted, and the third image captured with the third light emitted. The steps executed by the program are those shown in flowcharts of FIGS. 4-6, 10, and 12. As described above, the first light, the second light, and the third light have the following characteristics. That is, the first absorbance difference is smaller than the second absorbance difference, where the first absorbance difference is a difference between the absorbance of β-carotene at the peak wavelength of the first light and the absorbance of β-carotene at the peak wavelength of the second light, and the second absorbance difference is a difference between the absorbance of metmyoglobin at the peak wavelength of the first light and the absorbance of metmyoglobin at the peak wavelength of the second light. The peak wavelength of the third light differs from the peak wavelengths of the first light and the second light.

2.2 Highlighting Process and Display Image Generation Process

FIG. 4 is a flowchart explaining the processing by the endoscope apparatus 1. At the start of this processing, the control section 18 determines whether a current observation mode is a white light observation mode (S101). If the current observation mode is a white light observation mode (Yes at S101), the illumination section 3 sequentially lights the three LEDs respectively corresponding to the three kinds of light B1, G1, and R1 shown in FIG. 3A to cause these LEDs to sequentially emit the light B1, G1, and R1 (S102). The imaging section 10 uses the image sensor 12 to sequentially capture images using return light from an object corresponding to the respective kinds of emitted illumination light (S103). At S103, the imaging section 10 sequentially captures a B1 image based on emission of B1, a G1 image based on emission of G1, and the R1 image based on emission of R1, and these acquired images (image data or image information) are sequentially stored in the memory 16. Note that the order of emitting the three kinds of illumination light and the order of capturing the above images may be modified in various ways. The image processing section 17 performs an image processing corresponding to the white light observation mode based on the images stored in the memory 16 (S104).

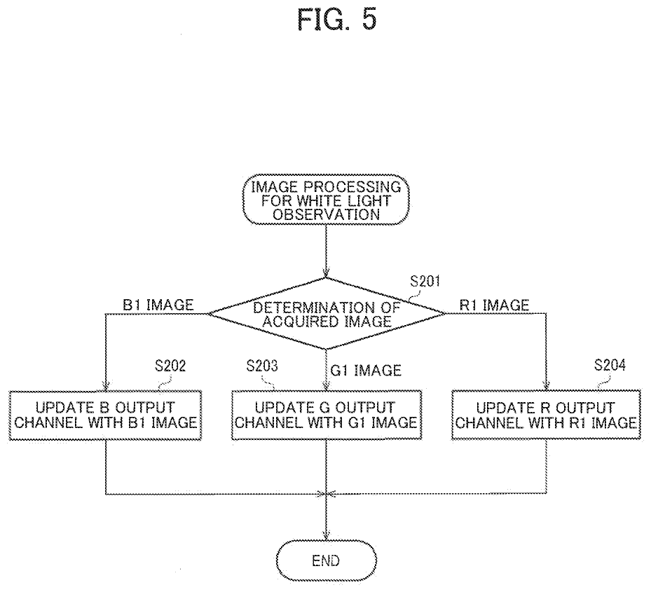

FIG. 5 is a flowchart explaining the processing at S104. The image processing section 17 determines whether an image acquired by the processing at S103 is the B1 image, the G1 image, or the R1 image (S201). If the acquired image is the B1 image, the image processing section 17 allocates the B1 image to the B output channel to update the display image (S202). Likewise, if the acquired image is the G1 image, the image processing section 17 allocates the G1 image to the G output channel (S203), and if the acquired image is the R1 image, the image processing section 17 allocates the R1 image to the R output channel (S204). On acquisition of the images corresponding to the three kinds of illumination light B1, G1, and R1, all three output channels have the respective images allocated, generating a white light image. Note that the white light image may be updated either per frame or per every three frames. The generated white light image is transmitted to the display section 6, which in turn displays the white light image.

As shown in FIGS. 3B and 3C, a region having myoglobin present has a higher absorption within the wavelength bands of B1 and G1 than within the wavelength band of R1. Thus, the region having myoglobin present is displayed in a pale reddish color in a white light image. Specifically, the mucosa layer, which has a higher myoglobin concentration, and the muscle layer, which has a lower myoglobin concentration, assume different colors, and the mucosa layer is displayed in a more reddish color while the muscle layer is displayed in a more whitish color.

A region having metmyoglobin present has a lower absorption of G1 than myoglobin. Thus, the region having metmyoglobin present is displayed in a yellowish color. A region having β-carotene present has extremely high absorption within the wavelength band of B1. Thus, the region having β-carotene present is displayed in a yellowish color.

Since both of the thermally denatured muscle layer rich in metmyoglobin and the fat layer rich in β-carotene are displayed in a yellowish color, it is difficult to identify these layers from each other. More specifically, it is difficult to identify the fat layer which can be an indicator of the risk of perforation.

Hence, the endoscope apparatus 1 operates of the present embodiment in a special light observation mode that is different from the white light observation mode. Note that the switch between the observation modes is made through the external I/F section 19, for example. The description will now return to FIG. 4. If a current observation mode is determined as the special light observation mode at S101 (No at S101), the illumination section 3 sequentially lights the four LEDs respectively corresponding to the four kinds of light B1, G2, G3, and R1 shown in FIG. 3B to cause these LEDs to sequentially emit the light B1, G2, G3, and R1 (S105). The imaging section 10 uses the image sensor 12 to sequentially capture images using return light from an object corresponding to the respective kinds of emitted illumination light (S106). At S106, the imaging section 10 sequentially captures the B1 image, the G2 image, the G3 image, and the R1 image, and these acquired images are sequentially stored in the memory 16. Note that the order of emitting the four kinds of illumination light and the order of capturing the above images may be modified in various ways. The image processing section 17 performs image processing corresponding to the special light observation mode based on the images stored in the memory 16 (S107).

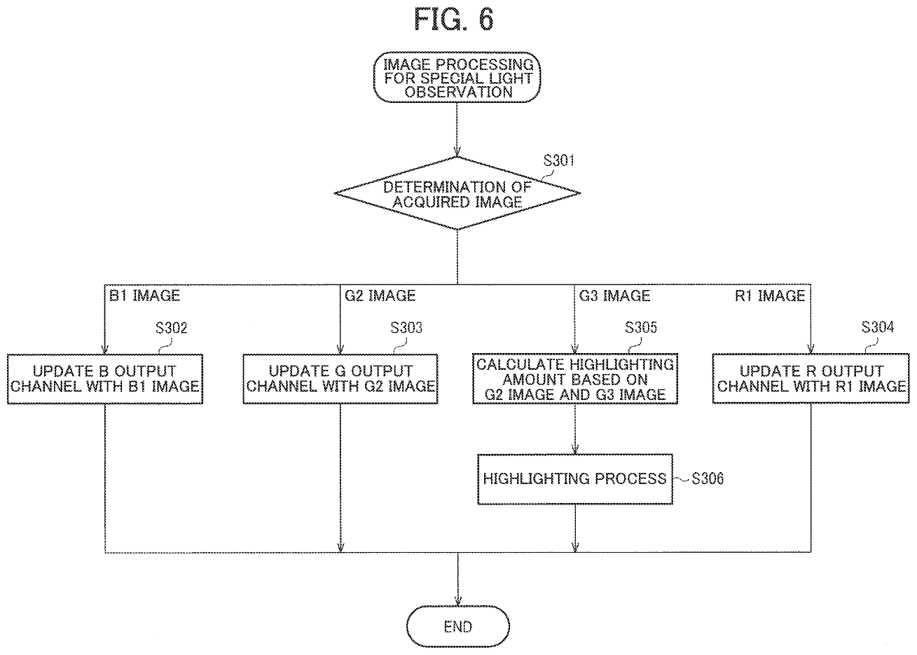

FIG. 6 is a flowchart explaining the processing at S107. The image processing section 17 determines whether an image acquired at S106 is the B1 image, the G2 image, the G3 image, or the R1 image (S301). If the acquired image is the B1 image, the image processing section 17 allocates the B1 image to the B output channel (S302). Likewise, if the acquired image is the G2 image, the image processing section 17 allocates the G2 image to the G output channel (S303), and if the acquired image is the R1 image, the image processing section 17 allocates the R1 image to the R output channel (S304).

If the acquired image is the G3 image, the highlighting amount calculation section 17a of the image processing section 17 calculates the highlighting amount based on the G3 image and the already acquired G2 image (S305). Then, the highlighting processing section 17b of the image processing section 17 performs a highlighting process on the display image based on the calculated highlighting amount (306). The highlighting process on the display image refers to a highlighting process on at least one of the B1 image, the G2 image and the R1 image allocated to the respective output channels.

FIG. 6 illustrates an example where the G2 image is allocated to the G output channel. This is because the overlap between the wavelength bands of G2 and G1 is greater than the overlap between the wavelength bands of G3 and G1, and thus the use of the G2 image is considered to improve color rendering properties of the display image. Instead, the G3 image may be allocated to the G output channel. While FIG. 6 illustrates an example where the highlighting amount calculation process and the highlighting process are performed at the timing when the G3 image is acquired, these processes may be performed at the timing when the G2 image is acquired. Alternatively, the highlighting amount calculation process and the highlighting process may be performed at both timings when the G2 image is acquired and when the G3 image is acquired.

As shown in FIGS. 3B and 3C, metmyoglobin has a higher absorbance within the wavelength band of G2 than within the wavelength band of G3. In addition, both of myoglobin and β-carotene have a small absorbance difference between G2 and G3. Hence, correlation between the G2 image and the G3 image is such that a region with relatively low correlation corresponds to a region containing a large amount of metmyoglobin and a region with relatively high correlation corresponds to a region containing a large amount of myoglobin or β-carotene.

Specifically, the highlighting amount calculation section 17a calculates the highlighting amount based on a ratio between signal values of the first image and the second image. This allows the highlighting amount calculation section 17a to obtain the correlation between the first image and the second image by a simple calculation. More specifically, the highlighting amount calculation section 17a calculates the highlighting amount using the following expression (1).

Emp(x,y)=G2(x,y)/G3(x,y) (1)

In the above expression (1), Emp is a highlighting amount image representing the highlighting amount, and (x, y) represents a position in the image. G2(x, y) represents a pixel value at (x, y) in the G2 image, and G3(x, y) represents a pixel value at (x, y) in the G3 image. The highlighting amount image Emp can be obtained by calculation of the above expression (1) for each (x, y). In other words, a highlighting amount is calculated per pixel, and the highlighting amount image Emp is an aggregate of the calculated highlighting amounts.

In the above expression (1), if Emp>1, the value of Emp is clipped to 1. As shown in FIGS. 3B and 3C, the absorbance of each pigment is higher in the wavelength band of G2 than in the wavelength band of G3. Since a pixel value of a given pixel in the G2 image is considered to smaller than a pixel value of the same given pixel in the G3 image, the value of Emp is normally Emp≤1. If Emp>1, that may be because an object in question is anything other than a living body, such as treatment tools, or may be because of noise. In this regard, clipping the value of Emp to the upper limit of 1 allows for calculation of the highlighting amount that enables stable highlighting of only the region containing metmyoglobin. Note that the highlighting amount of the present embodiment is not limited to the ratio itself shown in the above expression (1), and includes various information obtained based on the ratio. For example, the highlighting amount of the present embodiment includes the result of the above clipping process.

The highlighting processing section 17b performs a color conversion process on the display image based on the highlighting amount. Specifically, the highlighting processing section 17b adjusts a value of the R output channel using the following expression (2).

B′(x,y)=B(x,y)

G′(x,y)=G(x,y)

R′(x,y)=R(x,y)×Emp(x,y) (2)

In the above expression, B, G, and R represent images of the B channel, the G channel, and the R channel, respectively, before the highlighting process. In the present embodiment, B(x, y) represents a pixel value at (x, y) in the B1 image, G(x, y) represents a pixel value at (x, y) in the G2 image, and R(x, y) represents a pixel value at (x, y) in the R1 image. Also, B′, G′, and R′ represent images of the B channel, the G channel, and the R channel, respectively, after the highlighting process. Performing the highlighting process shown in the above expression (2) reduces red signal values in the region containing metmyoglobin.

As a result, the thermally denatured muscle layer containing a large amount of metmyoglobin is displayed in a greenish color. The region containing a large amount of myoglobin or β-carotene has little change in color. Thus, the mucosa layer and the muscle layer containing a large amount of myoglobin are displayed in a reddish or whitish color, and the fat layer containing a large amount of β-carotene is displayed in a yellowish color. As such, the method of the present embodiment allows a boundary between the muscle layer and the fat layer to be displayed in a highly visible manner even when the muscle layer may possibly undergo thermal denaturation during procedures. In particular, when applied to TUR-Bt, the method of the present embodiment helps avoid perforation of the bladder wall during resection of a tumor on the bladder.

2.3 Modifications

Some modifications will be given below.

2.3.1 Modifications to the Highlighting Amount Calculation Process and the Highlighting Process

In the above expression (1), the highlighting amount calculation section 17a calculates the highlighting amount based on the ratio between the G2 image and the G3 image. However, the highlighting amount calculation section 17a may calculate the highlighting amount based on a difference between signal values of the first image and the second image. Specifically, the highlighting amount calculation section 17a may calculate the highlighting amount using the following expression (3).

Emp(x,y){G3(x,y)−G2(x,y)}/G3(x,y) (3)

In the above expression (3), if Emp<0, the value of Emp is clipped to 0. Similarly to the example of the above expression (1), a pixel value of a given pixel in the G2 image is considered to be smaller than a pixel value of the same given pixel in the G3 image, and thus the value of Emp is normally 0≤Emp≤1. If Emp<0, that may be because an object in question is anything other than a living body, such as treatment tools, or may be because of noise. In this regard, clipping the value of Emp to the lower limit of 0 allows for calculation of the highlighting amount that enables stable highlighting of only the region containing metmyoglobin. Note that the highlighting amount in this modification is not limited to the difference itself, and includes various information obtained based on the difference. For example, the highlighting amount includes the result of the normalization process using G3(x, y) as in the above expression (3) and the result of the clipping process.

The highlighting amount obtained using the above expression (3) approaches 0 with increase in correlation between the images and approaches 1 with decrease in the correlation. Hence, in the case of using the highlighting amount image Emp of the above expression (3) for the process of reducing red signal values of the region containing a large amount of metmyoglobin (i.e., the region with low correlation between the images), the highlighting processing section 17b calculates the following expression (4).

B′(x,y)=B(x,y)

G′(x,y)=G(x,y)

R′(x,y)=R(x,y)×{1−Emp(x,y)} (4)

Through the process using the above expressions (3) and (4), the thermally denatured muscle layer containing a large amount of metmyoglobin is displayed in a greenish color, the mucosa layer and the muscle layer containing a large amount of myoglobin are displayed in a reddish or whitish color, and the fat layer containing a large amount of β-carotene is displayed in a yellowish color.

While the description has been given of the color conversion process of changing signal values of the R output channel as an example of the highlighting process, the highlighting process is not limited to this. For example, the highlighting processing section 17b may perform a color conversion process of changing signal values of the G output channel or signal values of the B output channel. Alternatively, the highlighting processing section 17b may perform a color conversion process of changing signal values of two or more output channels.

The highlighting processing section 17b may perform a chroma conversion process as the highlighting process. In the case of highlighting chroma, the highlighting processing section 17b may convert an RGB color space of a combined image into an HSV color space. The conversion into the HSV color space is made using the following expressions (5)-(9).

H(x,y)(G(x,y)−B(x,y))/(Max(RGB(x,y))−Min(RGB(x,y)))×60° (5)

H(x,y)(B(x,y)−R(x,y))/(Max(RGB(x,y))−Min(RGB(x,y)))×60°+1200 (6)

H(x,y)(R(x,y)−G(x,y))/(Max(RGB(x,y))−Min(RGB(x,y)))×60°+2400 (7)

S(x,y)=(Max(RGB(x,y))−Min(RGB(x,y)))/(Max(RGB(x,y)) (8)

V(x,y)=Max(RGB(x,y)) (9)

The expression (5) represents a hue H in the case where luminance values of an R image are the largest among B, G, and R images. The expression (6) represents the hue H in the case where luminance values of the G image are the largest among the B, G, and R images. The expression (7) represents the hue H in the case where luminance values of the B image are the largest among the B, G, and R images. In the above expressions (5)-(9), S represents chroma (i.e., saturation), and V represents brightness (i.e., value). Max(RGB(x, y)) represents a highest pixel value at a position (x, y) in the R, B, and G images, and Min(RGB(x, y)) represents a lowest pixel value at a position (x, y) in the R, B, and G images.

In the case of highlighting chroma, the highlighting processing section 17b converts the RGB color space into the HSV color space using the above expressions (5)-(9) and then changes chroma of the region containing metmyoglobin using the following expression (10).

S′(x,y)=S(x,y)×1/(Emp(x,y)) (10)

In the above expression (10), S′ represents chroma after the highlighting, and S represents chroma before the highlighting. As the highlighting amount Emp takes a value not less than 0 and not more than 1, the chroma after the highlighting takes a value larger than that before the highlighting.

After highlighting the chroma, the highlighting processing section 17b converts the HSV color space back into the RGB color space using the following expressions (11)-(20). Note that floor in the following expression (11) represents truncation.

h(x,y)=floor{H(x,y)/60} (11)

P(x,y)=V(x,y)×(1−S(x,y)) (12)

Q(x,y)=V(x,y)×(1−S(x,y)×(H(x,y)/60−h(x,y)) (13)

T(x,y)=V(x,y)×(1−S(x,y)×(1−H(x,y)/60+h(x,y)) (14)

If h(x,y)=0,

B(x,y)=P(x,y)

G(x,y)=T(x,y)

R(x,y)=V(x,y) (15)

If h(x,y)=1,

B(x,y)=P(x,y)

G(x,y)=V(x,y)

R(x,y)=Q(x,y) (16)

If h(x,y)=2,

B(x,y)=T(x,y)

G(x,y)=V(x,y)

R(x,y)=P(x,y) (17)

If h(x,y)=3,

B(x,y)=V(x,y)

G(x,y)=Q(x,y)

R(x,y)=P(x,y) (18)

If h(x,y)=4,

B(x,y)=V(x,y)

G(x,y)=P(x,y)

R(x,y)=T(x,y) (19)

If h(x,y)=5,

B(x,y)=Q(x,y)

G(x,y)=P(x,y)

R(x,y)=V(x,y) (20)

The highlighting processing section 17b may perform a hue conversion process. For example, the highlighting processing section 17b performs a hue conversion process by applying the highlighting amount image Emp to the hue H while maintaining values of the chroma S and the brightness V.

As described above, the highlighting process of the present embodiment may be any one of the processes that enable easy identification between the fat layer and the thermally denatured muscle layer, i.e., improve visibility of the boundary between the fat layer and the thermally denatured muscle layer, and thus the specific content of the highlighting process may be modified in various ways.

2.3.2 Modifications to the Illumination Light

The above description has been given of the case where the observation modes can be switched between the white light observation mode and the special light observation mode and the illumination section 3 emits the five kinds of illumination light B1, G1, R1, G2, and G3 as shown in FIGS. 3A and 3B.

In the aforementioned special light observation mode, the four LEDs respectively emitting the light B1, G2, G3, and R1 are used as shown in FIG. 3B. The light B1 corresponds to the blue wavelength band, and the light R1 corresponds to the red wavelength band. The light G2 is narrowband light within the green wavelength band. Hence, a display image with high color rendering properties can be generated by allocating the B1 image to the B output channel, allocating the G2 image to the G output channel, and allocating the R1 image to the R output channel.

However, generation of the display image with high color rendering properties is not essential in the present embodiment as the method of the present embodiment is at least required to display the fat layer and the thermally denatured muscle layer in an identifiable manner. For example, as a modification, emission of B1 or R1 may be omitted in the special light observation mode. In this case, for generation of a display image, the G3 image is allocated to an output channel to which an image captured by emission of the omitted light has been allocated, for example.

For example, in the case of omitting the LED that emits R1, the image processing section 17 allocates the B1 image to the B output channel, allocates the G2 image to the G output channel, and allocates the G3 image to the R output channel to thereby generate a display image. In the case of omitting the LED that emits B1, the image processing section 17 allocates the G3 image to the B output channel, allocates the G2 image to the G output channel, and allocates the R1 image to the R output channel to thereby generate a display image. The highlighting processing section 17b may perform the highlighting process either on the R channel similarly to the above example or on another channel, and may also perform the chroma conversion process or the hue conversion process. Note that the above correspondence between the three captured images and the three output channels is merely exemplary, and the image processing section 17 may differently allocate the captured images to the respective output channels to generate the display image.

In this case, the display image in the special light observation mode is a pseudo-color image, in which a surgical field appears much differently from that in the white light observation mode. In other words, using both of B1 and R1 is preferable in consideration of color rendering properties. However, sequentially emitting the illumination light from the LEDs causes positional displacement between captured images due to difference in capture timings. When both of B1 and R1 are used, one period consists of four frames. When either of B1 or R1 is omitted, one period consists of three frames. That is, omitting one of B1 and R1 is advantageous in terms of reducing the positional displacement.

The method of the present embodiment is aimed at enabling identification between the fat layer and the thermally denatured muscle layer, and thus the white light observation mode itself is not essential. Accordingly, the method may omit the steps of S101-S104 in FIG. 4 and the processing in FIG. 5, and may repeat the steps of S105 to S107 and the processing in FIG. 6. In this case, the LED for emitting G1 may be omitted, so that the illumination section 3 includes either four LEDs corresponding to B1, G2, G3, and R1 or three LEDs further excluding the LED corresponding to B1 or R1.

As described above, the illumination section 3 of the present embodiment emits at least the third light in addition to the first light (G2) and the second light (G3). The third light has a peak wavelength within the blue wavelength band or within the red wavelength band. The light with a peak wavelength within the blue wavelength band refers to light (B1) corresponding to the wavelength band of 450-500 nm. The light with a peak wavelength within the red wavelength band refers to light (R1) corresponding to the wavelength band of 600-650 nm. Here, the light corresponding to the wavelength band of 450-500 nm refers to light that has emission intensity at or above a predetermined threshold within the range of 450-500 nm. The same holds for other light corresponding to other wavelengths. Specifically, the third light has a wider wavelength band than that of the first light and that of the second light.

The first light and the second light of the present embodiment are useful for identification of whether an object in question is a region containing a large amount of metmyoglobin. With the first light and the second light alone, however, it is difficult to identify whether an object in question is a region containing a large amount of β-carotene or a region containing a large amount of myoglobin. In this regard, adding B1 or R1 enables identification between β-carotene and myoglobin.

For example, β-carotene has an extremely higher absorbance within the wavelength band of B1 than within the wavelength bands of G2 and G3. Hence, the fat layer can be displayed such that a color from the output channel to which the B1 image is input is muted and colors from the output channels to which the G2 image and the G3 image are input are dominant. Meanwhile, myoglobin has a lower absorbance within the wavelength band of B1 than within the wavelength bands of G2 and G3. Hence, the muscle layer and the mucosa layer can be displayed such that the color from the output channel to which the B1 image is input is relatively strong and the colors from the output channels to which the G2 image and the G3 image are input are relatively weak. This means that a combined display image that is generated from the input of the B1, G2, and G3 images to the respective channels can display the fat layer in a color different from a color of the muscle layer or the mucosa layer, allowing for easy identification between these layers.

The same holds for the case of adding R; a combined display image that is generated from the input of the G2, G3, and R1 images to the respective channels can display the fat layer in a color different from a color of the muscle layer or the mucosa layer.

In consideration of color rendering properties of the display image, it is preferable to emit fourth illumination light (hereinafter, fourth light) besides the third light. The fourth light is set to a visible wavelength band that is covered by none of the first to third light. Specifically, in the case where the third light has a peak wavelength within the blue wavelength band (i.e., B1), the illumination section 3 emits light with a peak wavelength within the red wavelength band (i.e., R1) as the fourth light. In the case where the third light has a peak wavelength within the red wavelength band (i.e., R1), the illumination section 3 emits light with a peak wavelength within the blue wavelength band (i.e., B1) as the fourth light.

This emission allows generation of a display image with high color rendering properties also in the special light observation mode.

2.3.3 Other Modifications

While the above example assumes that the image sensor 12 is a monochrome sensor, the image sensor 12 may be a color sensor including a color filter. Specifically, the image sensor 12 may be a color CMOS sensor or a color CCD sensor.

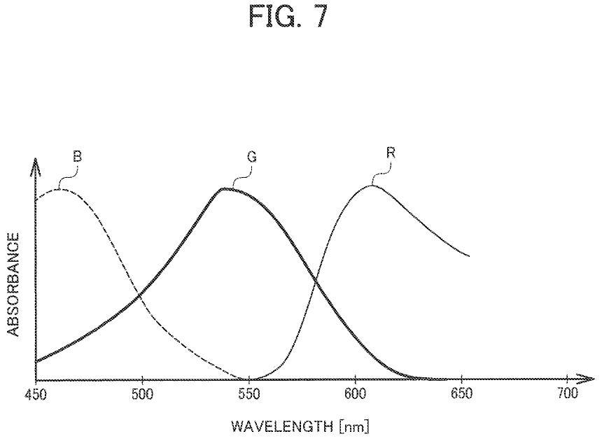

FIG. 7 illustrates an example of spectral characteristics of a color filter of the image sensor 12. The color filter includes three filters transmitting wavelength bands respectively corresponding to R, G, and B. The color filter may be a Bayer array filter, a filter having any other form of array, or a complementary filter.

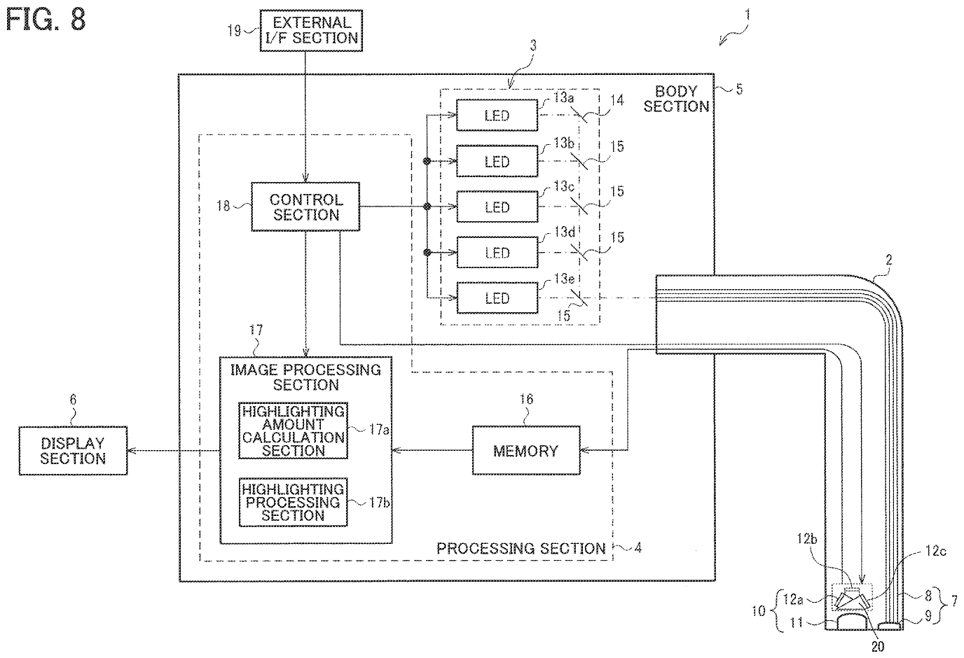

Alternatively, the image sensor 12 may be composed of a plurality of monochrome sensors. FIG. 8 illustrates another configuration example of the endoscope apparatus 1. The imaging section 10 of the endoscope apparatus 1 may include a color separation prism 20 that separates reflection light from an object into wavelength bands, and three image sensors 12a, 12b, and 12c that capture images of light in the respective wavelength bands separated by the color separation prism 20.

In the case where the image sensor 12 includes the color filter or is composed of the plurality of sensors (12a-12c), the illumination section 3 may simultaneously emit light in different wavelength bands and the imaging section 10 may capture images corresponding to the respective wavelength bands.

For example, in the white light observation mode, the illumination section 3 simultaneously lights the LEDs emitting B1, G1, and R1. The imaging section 10 simultaneously captures the B1 image, the G1 image, and the R1 image, enabling the while light observation.

In the special light observation mode, the illumination section 3 alternately lights a combination of the LEDs emitting B1 and G3 and a combination of the LEDs emitting G2 and R1, for example. The imaging section 10 captures a combination of the B1 image and the G3 image and a combination of the G2 image and the R1 image in a two-frame sequential method, enabling the special light observation. The above combinations are chosen in view of color separability, but other combinations are also possible except a combination that simultaneously emits G2 and G3.

While the above description has been given of the case where the respective kinds of light are emitted by the LEDs, laser diodes may replace the LEDs. In particular, the LEDs emitting narrowband light G2 and G3 may be replaced with laser diodes.

Also, the configuration of the illumination section 3 is not limited to one including the LEDs 13a-13e, the mirror 14, and the dichroic mirrors 15 as shown in FIG. 2. For example, the illumination section 3 may sequentially emit light within different wavelength bands by using a white light source for emitting white light, such as a Xenon lamp, and a filter turret including color filters each transmitting a wavelength band corresponding to each illumination light. In this case, the Xenon lamp may be replaced with a combination of a phosphor and a laser diode for exciting the phosphor.

In one contemplated form, the endoscope apparatus may include a control device and a scope connected to each other and may capture in-vivo images as a user operates the scope. Besides this, for example, a surgery support system and the like using a robot can also be contemplated as one form of the endoscope apparatus of the present embodiment.

For example, a surgery support system may include a control device, a robot, and a scope. The scope is a rigid scope, for example. The control device controls the robot. By operating an operation section of the control device, a user moves the robot and performs surgery on a patient using the robot. Also by operating the operation section of the control device, the user operates the scope via the robot and captures images of a surgical region. The control device may include the processing section 4 in FIG. 2. The user operates the robot while viewing images shown by the processing section 4 on the display device. The present embodiment may be applied to the control device in a surgery support system of this kind. The control device may be built into the robot.

### 3. Second Embodiment

Now a description will be given of a second embodiment. Similar configurations and processes to those in the first embodiment will be omitted from the description.

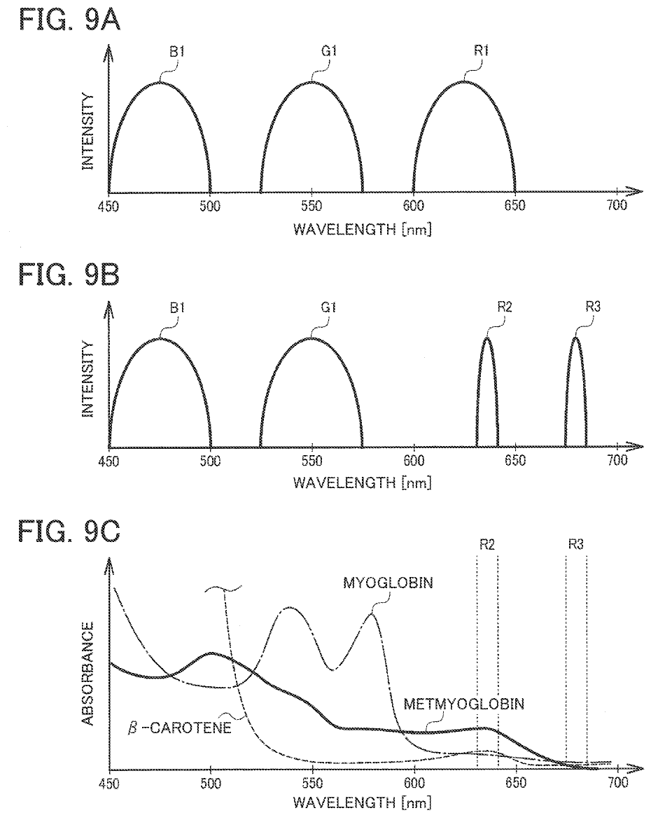

FIGS. 9A and 9B illustrate spectral characteristics of the plurality of LEDs 13a-13e. In FIGS. 9A and 9B, the horizontal axis represents wavelength, and the vertical axis represents intensity of the emitted light. The illumination section 3 of the second embodiment includes three LEDs respectively emitting the light B1 in the blue wavelength band, the light G1 in the green wavelength band, and the light R1 in the red wavelength band. Each wavelength band is similar to that in the first embodiment.

The illumination section 3 of the present embodiment further includes two LEDs respectively emitting narrowband light R2 and R3 within the red wavelength band. In the present embodiment, the first light corresponds to R2 and the second light corresponds to R3. That is, the first light is narrowband light with a peak wavelength within a range of 630 nm±10 nm, and the second light is narrowband light with a peak wavelength within a range of 680 nm±10 nm.

FIG. 9C illustrates absorption characteristics of β-carotene, metmyoglobin, and myoglobin, which is similar to FIG. 3C.

If R2 and R3 are set to the wavelengths shown in FIG. 9B, the absorbance of β-carotene within the wavelength band of R2 is substantially the same as the absorbance of β-carotene within the wavelength band of R3, and the absorbance of myoglobin within the wavelength band of R2 is substantially the same as the absorbance of myoglobin within the wavelength band of R3. Hence, in a region containing β-carotene or myoglobin, there is a small difference between signal values of an R2 image obtained by emission of R2 and an R3 image obtained by emission of R3.

On the other hand, metmyoglobin has a higher absorbance within the wavelength band of R2 than within the wavelength band of R3. Hence, in a region containing metmyoglobin, signal values of the R2 image obtained by emission of R2 are smaller than those of the R3 image obtained by emission of R3, so that the R2 image is darker than the R3 image in that region.

The processing by the endoscope apparatus 1 of the present embodiment is similar to that shown in FIG. 4. Also, the processing in the white light observation mode is similar to that shown in FIG. 5. In the white light observation mode, the illumination section 3 sequentially lights the three LEDs respectively corresponding to the three kinds of light B1, G1, and R1 shown in FIG. 9A to cause these LEDs to sequentially emit the light B1, G1, and R1 (102). The imaging section 10 uses the image sensor 12 to sequentially capture images using return light from an object corresponding to the respective kinds of emitted illumination light (S103). The image processing section 17 allocates the B1 image to the B output channel, the G1 image to the G output channel, and the R1 image to the R output channel (S104 and FIG. 5).

If a current observation mode is determined as the special light observation mode, the illumination section 3 sequentially lights the four LEDs respectively corresponding to the four kinds of light B1, G1, R2, and R3 shown in FIG. 9B to cause these LEDs to sequentially emit the light B1, G1, R2, and R3 (S105). The imaging section 10 uses the image sensor 12 to sequentially capture images using return light from an object corresponding to the respective kinds of emitted illumination light (S106). At S106 in the second embodiment, the imaging section 10 sequentially captures the B1 image, the G1 image, the R2 image, and the R3 image, and these acquired images are sequentially stored in the memory 16.

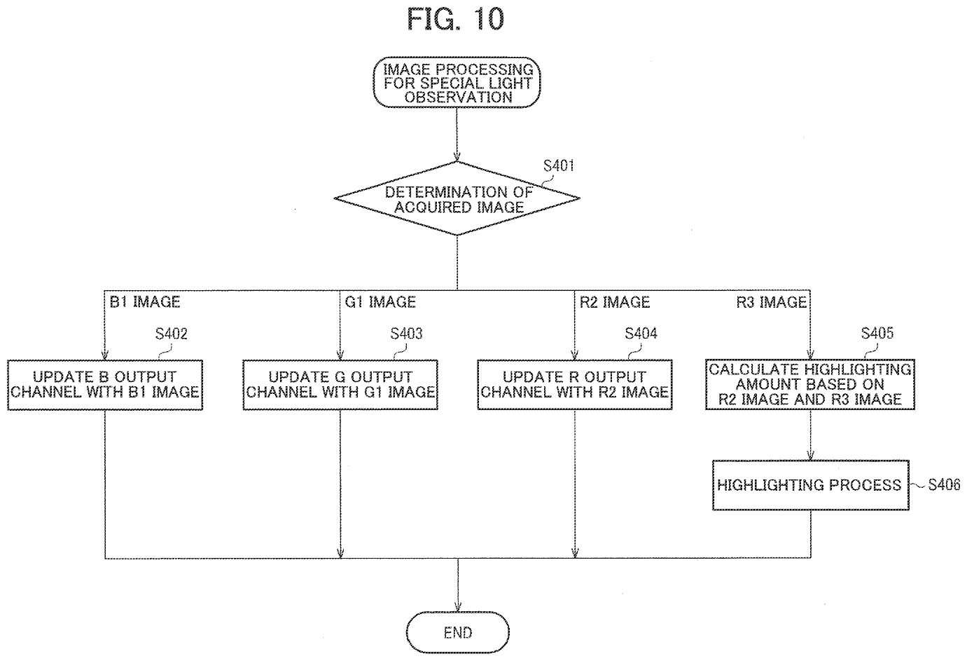

FIG. 10 is a flowchart explaining the processing at S107 in the second embodiment. The image processing section 17 determines whether an image acquired at S106 is the B1 image, the G1 image, the R2 image, or the R3 image (S401). If the acquired image is the B1 image, the image processing section 17 allocates the B1 image to the B output channel (S402). Likewise, if the acquired image is the G1 image, the image processing section 17 allocates the G1 image to the G output channel (S403), and if the acquired image is the R2 image, the image processing section 17 allocates the R2 image to the R output channel (S404).

If the acquired image is the R3 image, the highlighting amount calculation section 17a of the image processing section 17 calculates the highlighting amount based on the R3 image and the already acquired R2 image (S405). Then, the highlighting processing section 17b of the image processing section 17 performs the highlighting process on the display image based on the calculated highlighting amount (S406).

The light R2, R3 and the light G2, G3 have similar characteristics regarding absorbance difference of each pigment, i.e., β-carotene, metmyoglobin, and myoglobin. Thus, the highlighting amount calculation section 17a calculates the highlighting amount using the following expression (21) or (22), similar to the above expression (1) or (3).

Emp(x,y)=R2(x,y)/R3(x,y) (21)

Emp(x,y){R3(x,y)−R2(x,y)}/R3(x,y) (22)

The highlighting processing section 17b may perform the highlighting process using either the above expression (2) or (4). Alternatively, various modifications are possible as described above, including performing the process of converting signal values of channels other than the R output channel, the chroma conversion process, and the hue conversion process.

FIG. 10 illustrates an example where the R2 image is allocated to the R output channel. This is because the overlap between the wavelength bands of R2 and R1 is greater than the overlap between the wavelength bands of R3 and R1, and thus the use of the R2 image is considered to improve color rendering properties of the display image. Instead, the R3 image may be allocated to the R output channel. While FIG. 10 illustrates an example where the highlighting amount calculation process and the highlighting process are performed at the timing when the R3 image is acquired, these processes may be performed at the timing when the R2 image is acquired. Alternatively, the highlighting amount calculation process and the highlighting process may be performed at both timings when the R2 image is acquired and when the R3 image is acquired.

The modifications given in the first embodiment may also be applied to the present embodiment. That is, both of B1 and G1 may be used in consideration of color rendering properties, or one of them may be omitted so as to display a pseudo-color image.

The third light in the second embodiment has a peak wavelength within the blue wavelength band or within the green wavelength band. The light with a peak wavelength within the blue wavelength band refers to light (B1) corresponding to the wavelength band of 450-500 nm. The light with a peak wavelength within the green wavelength band refers to light (G) corresponding to the wavelength band of 525-575 nm. Specifically, the third light has a wider wavelength band than that of the first light and that of the second light. Adding B1 or G1 enables identification between β-carotene and myoglobin. Specifically, a combined display image that is generated from the input of the B1, R2, and R3 images to the respective channels can display the fat layer in a color different from a color of the muscle layer or the mucosa layer. Alternatively, a combined display image that is generated from the input of the G1, R2, and R3 images to the respective channels can display the fat layer in a color different from a color of the muscle layer or the mucosa layer.

In the case where the third light has a peak wavelength within the blue wavelength band (i.e., B1), the illumination section 3 may further emit light with a peak wavelength within the green wavelength band (i.e., G1) as the fourth light. Alternatively, in the case where the third light has a peak wavelength within the green wavelength band (i.e., G1), the illumination section 3 may further emit light with a peak wavelength within the blue wavelength band (i.e., B1) as the fourth light. This emission allows generation of a display image with high color rendering properties also in the special light observation mode.

Similarly to the first embodiment, various modifications may be made to the image sensor 12 and the illumination section 3.

### 4. Third Embodiment

The first and second embodiments are directed to the case where the absorbance of myoglobin at the peak wavelength of the first light is substantially the same as the absorbance of myoglobin at the peak wavelength of the second light. In this case, the use of the first image and the second image enables identification of whether a pigment present in large quantity in an object is metmyoglobin, or otherwise β-carotene or myoglobin. In other words, while images of various objects including the fat layer, the thermally denatured muscle layer, the muscle layer, and the mucosa layer are captured during image capture, this method can focus the highlighting process on the thermally denatured muscle layer among these layers.

However, if any alternative method enables identification between metmyoglobin and myoglobin, the first light and the second light are only required to satisfy the condition that the first absorbance difference is smaller than the second absorbance difference. In other words, with such a method, relation between the absorbance of the first light by myoglobin and the absorbance of the second light by myoglobin may be set in any ways.

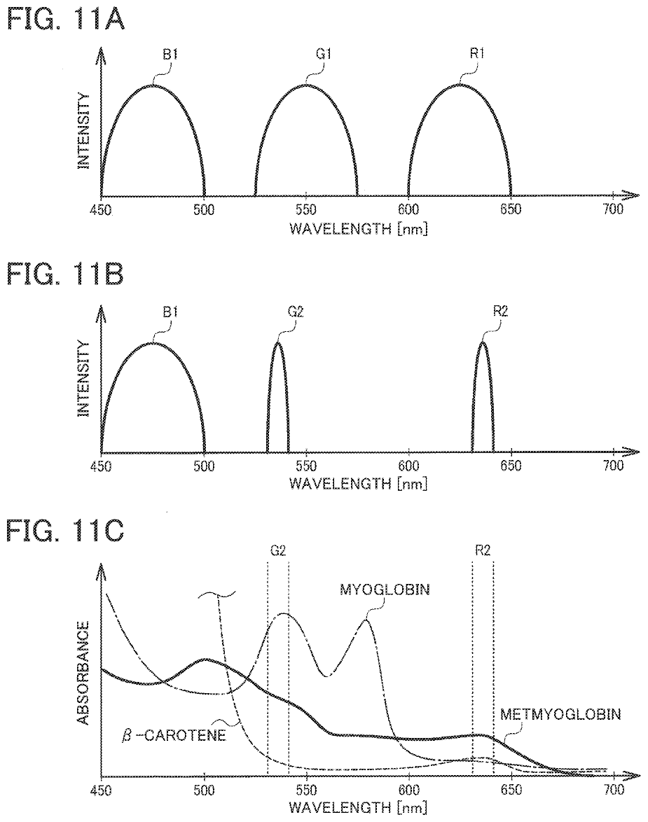

FIGS. 11A and 11B illustrate spectral characteristics of a plurality of LEDs. In FIGS. 11A and 11B, the horizontal axis represents wavelength, and the vertical axis represents intensity of the emitted light. The illumination section 3 of the third embodiment includes three LEDs respectively emitting the light B1 in the blue wavelength band, the light G1 in the green wavelength band, and the light R1 in the red wavelength band. Each wavelength band is similar to that in the first embodiment.

The illumination section 3 of the present embodiment further includes two LEDs respectively emitting the narrowband light G2 within the green wavelength band and the narrowband light R2 within the red wavelength band.

The absorbance of β-carotene within the wavelength band of G2 is substantially the same as the absorbance of β-carotene within the wavelength band of R2. Hence, in a region containing β-carotene, there is a small difference between signal values of the G2 image obtained by emission of G2 and the R2 image obtained by emission of R2.

On the other hand, metmyoglobin has a higher absorbance within the wavelength band of G2 than within the wavelength band of R2. Hence, in a region containing metmyoglobin, signal values of the G2 image obtained by emission of G2 are smaller than those of the R2 image obtained by emission of R2, so that the G2 image is darker than the R2 image in that region.

Thus, with the highlighting amount calculated by the following expression (23) for example, it is possible to increase a change in the signal values in the thermally denatured muscle layer region, which contains a large amount of metmyoglobin, while giving a small change in the signal values in the fat layer region, which contains a large amount of β-carotene.

Emp(x,y)=G2(x,y)/R2(x,y) (23)

However, myoglobin has a higher absorbance within the wavelength band of G2 than within the wavelength band of R2. Thus, the highlighting process using the highlighting amount Emp obtained by the above expression (23) also causes unwanted great changes in the signal values of the region containing a large amount of myoglobin, more specifically the muscle layer and the mucosa layer.

In view of this, the method of the present embodiment detects a region that is determined as either the fat layer or the thermally denatured muscle layer, from captured images. The highlighting processing section 17b performs the highlighting process using the above highlighting amount only on that detected region. Thus, the method can avoid unnecessary highlighting processes by excluding the region containing a large amount of myoglobin through the detection process.

The processing by the endoscope apparatus 1 of the present embodiment is similar to that shown in FIG. 4. Also, the processing in the white light observation mode is similar to that shown in FIG. 5.

If a current observation mode is determined as the special light observation mode, the illumination section 3 sequentially lights the three LEDs respectively corresponding to the three kinds of light B1, G2, and R2 shown in FIG. 11B to cause these LEDs to sequentially emit the light B1, G2, and R2 (105). The imaging section 10 uses the image sensor 12 to sequentially capture images using return light from an object corresponding to the respective kinds of emitted illumination light (S106). At S106 in the third embodiment, the imaging section 10 sequentially captures the B1 image, the G2 image, and the R2 image, and these acquired images are sequentially stored in the memory 16.

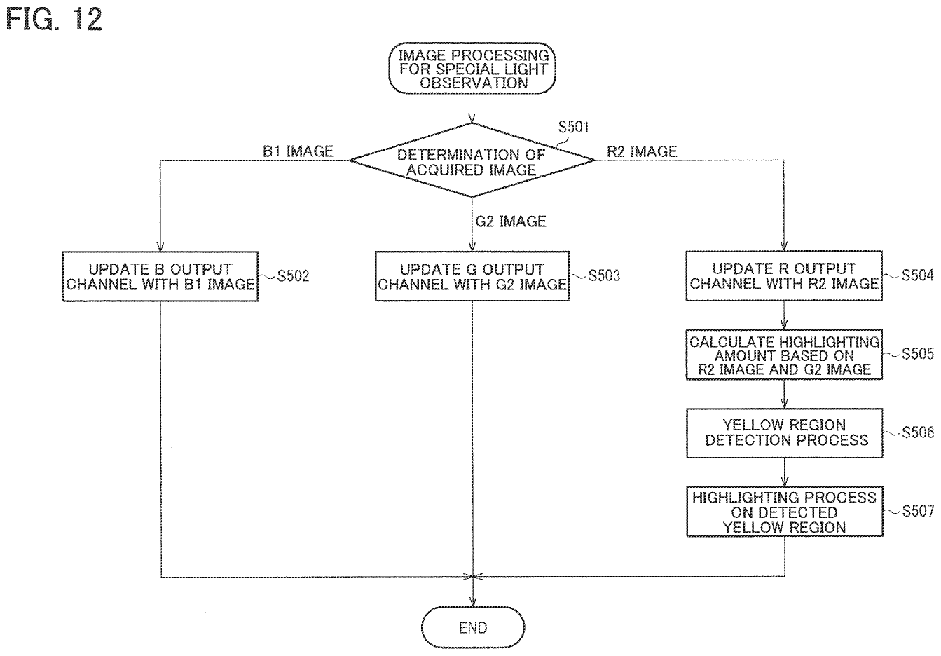

FIG. 12 is a flowchart explaining the processing at S107 in the third embodiment. The image processing section 17 determines whether an image acquired at S106 is the B1 image, the G2 image, or the R2 image (S501). If the acquired image is the B1 image, the image processing section 17 allocates the B1 image to the B output channel (S502). Likewise, if the acquired image is the G2 image, the image processing section 17 allocates the G2 image to the G output channel (S503), and if the acquired image is the R2 image, the image processing section 17 allocates the R2 image to the R output channel (S504).

If the acquired image is the R2 image, the highlighting amount calculation section 17a of the image processing section 17 calculates the highlighting amount based on the R2 image and the already acquired G2 image (S505). The image processing section 17 further performs a color determination process on a display image before the highlighting process to detect a region that is determined as a yellow region (506). For example, the image processing section 17 obtains color differences Cr, Cb based on signal values of the respective RGB channels and detects a region whose Cr and Cb are within a predetermined range as a yellow region.

The light G2 corresponds to the green wavelength band, and the light R2 corresponds to the red wavelength band. Thus, allocating the B1 image, the G2 image, and the R2 image respectively to the B output channel, the G output channel, and the R output channel can increase color rendering properties of the display image to some extent. As a result, the fat layer and the thermally denatured muscle layer are displayed in a yellowish color while the muscle layer and the mucosa layer are displayed in a reddish or whitish color. This means that, through detection of a region of a predetermined color based on images allocated to the respective channels in the special light observation mode, it is possible to detect a region that is estimated to be either the fat layer or the thermally denatured muscle layer.

At S507, the highlighting processing section 17b of the image processing section 17 performs the highlighting process based on the highlighting amount calculated at S505, on the yellow region detected at S506.

Similarly to the first and second embodiments, the method of the present embodiment allows for display of the fat layer and the thermally denatured muscle layer in an easily identifiable manner. Comparing the three embodiments, an advantage in the first and second embodiments is to be able to perform the highlighting process on an entire captured image without the process of detecting the yellow region, thereby imposing less processing load. Meanwhile, an advantage in the third embodiment is to be able to set wavelength bands of the first light and the second light without considering the absorbance of myoglobin, thereby allowing for flexibility in setting wavelength bands.

While the above description has been given of the case where the image processing section 17 detects the yellow region, various modifications are possible such that e.g., the image processing section 17 detects a red region and a white region and performs the highlighting process on regions other than these detected regions in a captured image.

While the above description has been given of the case where the first light is G2 and the second light is R2, various modifications may be made to the specific wavelength bands of the light. The only requirement in the above embodiment is that the first absorbance difference of β-carotene is smaller than the second absorbance difference of metmyoglobin.

Also, various modifications may be made to the wavelength band of the third light. For example, the third light is not limited to B1, and only needs to have a visible wavelength band covered by neither the first light nor the second light. While the above description has been given of the case of generating a display image with high color rendering properties using the first to third light, the embodiment may be modified to generate a pseudo-color image based on the first to third light. In this case, the fourth light may be added to increase the color rendering properties of the display image.

Similarly to the first and second embodiments, various modifications may be made to the highlighting amount calculation process and the highlighting process and also to the image sensor 12 and the illumination section 3.

Although the embodiments to which the present disclosure is applied and the modifications thereof have been described in detail above, the present disclosure is not limited to the embodiments and the modifications thereof, and various modifications and variations in components may be made in implementation without departing from the spirit and scope of the present disclosure. The plurality of elements disclosed in the embodiments and the modifications described above may be combined as appropriate to implement the present disclosure in various ways. For example, some of all the elements described in the embodiments and the modifications may be deleted. Furthermore, elements in different embodiments and modifications may be combined as appropriate. Thus, various modifications and applications can be made without departing from the spirit and scope of the present disclosure. Any term cited with a different term having a broader meaning or the same meaning at least once in the specification and the drawings can be replaced by the different term in any place in the specification and the drawings.

What is claimed is:

1. An endoscope apparatus comprising:
an illumination device emitting first illumination light, second illumination light, and third illumination light;
an imaging device capturing an image using return light, from a subject, based on light emitted from the illumination device; and
a processor including hardware,
the processor being configured to generate a display image on the basis of a first image captured with the first illumination light emitted, a second image captured with the second illumination light emitted, and a third image captured with the third illumination light emitted,
a first absorbance difference being smaller than a second absorbance difference, the first absorbance difference being a difference between an absorbance of β-carotene at a peak wavelength of the first illumination light and an absorbance of β-carotene at a peak wavelength of the second illumination light, the second absorbance difference being a difference between an absorbance of metmyoglobin at the peak wavelength of the first illumination light and an absorbance of metmyoglobin at the peak wavelength of the second illumination light,
a peak wavelength of the third illumination light differing from the peak wavelength of the first illumination light and the peak wavelength of the second illumination light,
the processor generating, based on the first image, the second image, and the third image, the display image that displays a thermally denatured muscle layer of the subject, a fat layer of the subject, and a muscle layer of the subject that is not thermally denatured, in a manner allowing for identification of the layers from each other.

2. The endoscope apparatus as defined in claim 1,
a third absorbance difference being smaller than the second absorbance difference, the third absorbance difference being a difference between an absorbance of myoglobin at the peak wavelength of the first illumination light and an absorbance of myoglobin at the peak wavelength of the second illumination light.

3. The endoscope apparatus as defined in claim 1,
the first illumination light being narrowband light with a peak wavelength within a range of 540 nm±10 nm,
the second illumination light being narrowband light with a peak wavelength within a range of 580 nm±10 nm.

4. The endoscope apparatus as defined in claim 3,
the third illumination light being light with a peak wavelength within a blue wavelength band or light with a peak wavelength within a red wavelength band.

5. The endoscope apparatus as defined in claim 4,
the illumination device emitting fourth light, the fourth light being:
(i) light with a peak wavelength within the red wavelength band, in a case where the third illumination light is light with a peak wavelength within the blue wavelength band; and
(ii) light with a peak wavelength within the blue wavelength band, in a case where the third illumination light is light with a peak wavelength within the red wavelength band.

6. The endoscope apparatus as defined in claim 4,
the light with a peak wavelength within the blue wavelength band being light that corresponds to a wavelength band of 450-500 nm,
the light with a peak wavelength within the red wavelength band being light that corresponds to a wavelength band of 600-650 nm.

7. The endoscope apparatus as defined in claim 1,
the first illumination light being narrowband light with a peak wavelength within a range of 630 nm±10 nm,
the second illumination light being narrowband light with a peak wavelength within a range of 680 nm±10 nm.

8. The endoscope apparatus as defined in claim 7,
the third illumination light being light with a peak wavelength within a blue wavelength band or light with a peak wavelength within a green wavelength band.

9. The endoscope apparatus as defined in claim 8,
the illumination device emitting fourth light, the fourth light being:
(i) light with a peak wavelength within the green wavelength band, in a case where the third illumination light is light with a peak wavelength within the blue wavelength band; and
(ii) light with a peak wavelength within the blue wavelength band in a case where the third illumination light is light with a peak wavelength within the green wavelength band.

10. The endoscope apparatus as defined in claim 8,
the light with a peak wavelength within the blue wavelength band being light that corresponds to a wavelength band of 450-500 nm,
the light with a peak wavelength within the green wavelength band being light that corresponds to a wavelength band of 525-575 nm.

11. The endoscope apparatus as defined in claim 1,
the processor calculating a highlighting amount based on correlation between the first image and the second image,
the processor performing a highlighting process on the display image based on the highlighting amount.

12. The endoscope apparatus as defined in claim 11,
the processor calculating the highlighting amount based on a ratio or a difference between a signal value of the first image and a signal value of the second image.

13. The endoscope apparatus as defined in claim 11,
the processor performing a color conversion process on the display image based on the highlighting amount.

14. The endoscope apparatus as defined in claim 1,
the subject being a bladder wall.

15. An operation method of an endoscope apparatus, the method comprising:
emitting first illumination light, second illumination light, and third illumination light;
capturing an image using return light, from a subject, based on emission of the first illumination light, the second illumination light, and the third illumination light; and
generating a display image on the basis of a first image captured with the first illumination light emitted, a second image captured with the second illumination light emitted, and a third image captured with the third illumination light emitted,
a first absorbance difference being smaller than a second absorbance difference, the first absorbance difference being a difference between an absorbance of β-carotene at a peak wavelength of the first illumination light and an absorbance of β-carotene at a peak wavelength of the second illumination light, the second absorbance difference being a difference between an absorbance of metmyoglobin at the peak wavelength of the first illumination light and an absorbance of metmyoglobin at the peak wavelength of the second illumination light,
a peak wavelength of the third illumination light differing from the peak wavelength of the first illumination light and the peak wavelength of the second illumination light,
the generating the display image comprising generating, based on the first image, the second image, and the third image, the display image that displays a thermally denatured muscle layer of the subject, a fat layer of the subject, and a muscle layer of the subject that is not thermally denatured, in a manner allowing for identification of the layers from each other.

16. A non-transitory information storage medium storing a program, the program causing a computer to execute steps comprising:
causing an illumination device to emit first illumination light, second illumination light, and third illumination light;
capturing an image using return light, from a subject, based on light emitted from the illumination device; and
generating a display image on the basis of a first image captured with the first illumination light emitted, a second image captured with the second illumination light emitted, and a third image captured with the third illumination light emitted,
a first absorbance difference being smaller than a second absorbance difference, the first absorbance difference being a difference between an absorbance of β-carotene at a peak wavelength of the first illumination light and an absorbance of β-carotene at a peak wavelength of the second illumination light, the second absorbance difference being a difference between an absorbance of metmyoglobin at the peak wavelength of the first illumination light and an absorbance of metmyoglobin at the peak wavelength of the second illumination light,
a peak wavelength of the third illumination light differing from the peak wavelength of the first illumination light and the peak wavelength of the second illumination light,
the step of generating the display image comprising generating, based on the first image, the second image, and the third image, the display image that displays a thermally denatured muscle layer of the subject, a fat layer of the subject, and a muscle layer of the subject that is not thermally denatured, in a manner allowing for identification of the layers from each other.
